(19) United States
(12) Patent Application Publication
(10) Pub. No.: US 2021/0282644 A1
(43) Pub. Date: Sep. 16, 2021

(54) CAP-BASED TRANSCRANIAL OPTICAL TOMOGRAPHY

(71) Applicant: BOARD OF REGENTS OF THE UNIVERSITY OF TEXAS SYSTEM, Austin, TX (US)

(72) Inventors: Manish N. Shah, Houston, TX (US); Banghe Zhu, Houston, TX (US); Eva M. Sevick, Houston, TX (US)

(21) Appl. No.: 17/197,607

(22) Filed: Mar. 10, 2021

Related U.S. Application Data
(60) Provisional application No. 62/987,447, filed on Mar. 10, 2020.

(51) Int. Cl. A61B 5/00

(52) U.S. Cl. CPC A61B 5/0073; A61B 5/0077; A61B 5/6803; A61B 5/0042

(57) ABSTRACT

A cap-based Transcranial Optical Tomography (CTOT) imaging system includes a cap, a first laser, a second laser, a first optical fiber, a second optical fiber, an intensifier, a third optical fiber, a fourth optical fiber, and an image sensor. The cap is configured for placement on a head of a subject to be imaged. The first laser source configured to generate a laser light at a first wavelength. The second laser source configured to generate laser light at a second wavelength. The first optical fiber and the second optical fiber couple the first laser source and the second laser to the cap. The third optical fiber couples the image intensifier to the cap. The fourth optical fiber couples the image intensifier to the cap. The image sensor is coupled to the image intensifier, and is configured to capture an image of intensified light generated by the image intensifier.

### CROSS-REFERENCE TO RELATED APPLICATION

The present application claims priority to U.S. Provisional Patent Application No. 62/987,447, filed Mar. 10, 2020, entitled “Cap-Based Transcranial Optical Tomography,” which is hereby incorporated herein by reference in its entirety.

### BACKGROUND

A variety of imaging technologies can be used to investigate brain function. Examples of such imaging technologies include functional magnetic resonance imaging (fMRI), electroencephalography (EEG), magnetoencephalography (MEG), positron emission tomography (PET), and functional near-infrared spectroscopy (fNIRS). EEG and MEG, rely on the detection of electrical signals in the brain, while the fMRI, PET, and fNIRS aim to detect the hemodynamic changes related to areas of activation. These techniques have generated vast amounts of new information regarding fundamental neural circuits over the last century, and are useful in many fields of research.

### SUMMARY

A cap-based Transcranial Optical Tomography (CTOT) imaging system is disclosed herein. In one example, a cap-based Transcranial Optical Tomography (CTOT) imaging system includes a cap, a first laser, a second laser, a first optical fiber, a second optical fiber, an intensifier, a third optical fiber, a fourth optical fiber, and an image sensor. The cap is configured to cover a head of a subject to be imaged. The first laser source configured to generate a laser light at a first wavelength. The second laser source configured to generate laser light at a second wavelength. The first optical fiber and the second optical fiber couple the first laser source and the second laser to the cap. The third optical fiber couples the image intensifier to the cap. The fourth optical fiber couples the image intensifier to the cap. The image sensor is coupled to the image intensifier, and is configured to capture an image of intensified light generated by the image intensifier.

In another example, a method for CTOT includes placing a cap on a subject to be imaged. A laser source, of a plurality of available laser sources, is selected to generate laser light. A source fiber or a plurality of available source fibers is selected to route the laser light to the cap. A mask (which may act as a shutter) is dynamically positioned to filter light received from the cap from one or more collection fibers positioned within a predetermined distance of the source fiber routing laser light to the cap. Light received from the cap through a first collection fiber positioned within a predetermined distance of the source fiber routing laser light to the cap is filtered. An image of light received from the cap through a second collection fiber positioned more than the predetermined distance from the source fiber routing laser light to the cap is captured.

Because the light collected will have a wide range of fluences that may be greater than the dynamic range of the sensor, the mask attenuates the brightest fluences so that they can be measured within the dynamic range of the sensor. The mask function can dynamically change depending upon which source fiber is used to deliver light. The mask is positioned to attenuate light coming from collection fibers closest to the source fiber that routes incident light. An image of light passing through the mask is captured and multiple images can be made with differing positions of the mask to collect light signals.

### BRIEF DESCRIPTION OF THE DRAWINGS

For a detailed description of various examples, reference will now be made to the accompanying drawings in which:

FIG. 1 shows a schematic level diagram of an example Cap-based Transcranial Optical Tomography (CTOT) imaging system in accordance with the present disclosure.

FIG. 2 shows an example of placement of source and detector fibers on a cap covering a subject to be imaged in accordance with the present disclosure.

FIG. 3 shows an example of images of a coupling plate depicting fluence from the collection fibers with illumination at two different wavelengths in accordance with the present disclosure.

FIG. 4 shows an example of cap including source and collection fibers disposed for imaging in accordance with the present disclosure.

FIGS. 5A-5D show a selection of images generated by functional magnetic resonance imaging (fMRI) and CTOT in accordance with the present disclosure.

FIG. 6 shows a flow diagram for a method for CTOT in accordance with the present disclosure.

FIG. 7 shows application of a digital micromirror device (DMD) in the CTOT imaging system of FIG. 1.

### DETAILED DESCRIPTION

Certain terms have been used throughout this description and claims to refer to particular system components. As one skilled in the art will appreciate, different parties may refer to a component by different names. This document does not intend to distinguish between components that differ in name but not function. In this disclosure and claims, the terms “including” and “comprising” are used in an open-ended fashion, and thus should be interpreted to mean “including, but not limited to . . . ” Also, the term “couple” or “couples” is intended to mean either an indirect or direct connection. Thus, if a first device couples to a second device, that connection may be through a direct connection or through an indirect connection via other devices and connections. The recitation “based on” is intended to mean “based at least in part on.” Therefore, if X is based on Y, X may be a function of Y and any number of other factors.

Functional neuroimaging, while of great utility of in adults, has many limitations in infants. Positron Emission Tomography (PET) scans require ionizing radiopharmaceuticals. Perfusion Computed Tomography (CT) provides low-resolution three-dimensional (3-D) hemodynamic brain imaging but involves ionizing radiation. In contrast, Blood Oxygen Level Dependent functional Magnetic Resonance Imaging (BOLD fMRI) is safe, non-invasive and does not use ionizing radiation. However, like PET and CT, with BOLD fMRI the subject needs to remain supine and still in order to prevent motion artifacts. Consequently, all of these imaging techniques are impractical in unsedated young children. In addition, PET/CT and fMRI techniques are limited by their temporal resolution and report neuronal activity averaged over 6-10 minutes, preventing evaluation of the complex, transient neurovascular coupling function.

Functional Near-Infrared (NIR) Spectroscopy (fNIRS) imaging is a promising brain-imaging technique. Imaging with fNIRS allows examination of brain metabolism that is comparable to the BOLD fMRI signal. The fNIRS signal maps total hemoglobin (HbT) as well as oxygenated hemoglobin (HbO) and de-oxygenated hemoglobin (HbR). This map approximates brain activation and deactivation acting as a proxy for localized glucose metabolism, similar to BOLD fMRI. Diffuse optical tomography (DOT) is an extension of fNIRS that combines multi-channel data acquisition with imaging reconstruction techniques to provide images of neural related hemodynamic changes. Brain DOT could radically alter our understanding of dysfunction in the pediatric brain, a critically important step to positively modifying disabling movement disorders associated with cerebral palsy and devastating cognitive dysfunction associated with childhood-onset epilepsy.

Despite being safe and well tolerated in neonates and children, conventional fNIRS-DOT has some limitations arising from the number of optical sources and detectors, the detector sensitivities, and the field-of-view. For example, conventional fNIRS-DOT systems generally are unable to measure the entire brain due to impaired detector sensitivity or sparse number of collection fibers. In addition, all fNIRS-DOT systems have multiple detectors which are dedicated to each collection fiber or a group of collection fibers. The number of detectors needed can limit the number of measurements taken further restricting measurement of the entire brain. If whole brain measurement is provided, then systems generally require an acquisition time that is too long to evaluate transient neurovascular coupling functions.

The systems disclosed herein provide fNIRS-DOT imaging that addresses the various limitations of convention brain imaging techniques. The present disclosure presents a whole brain Cap-based Transcranial Optical Tomography (CTOT) imaging system based on a single, GaAs intensified integrating detector array that enables sensitive detection of diffuse near-infrared (NIR) light in transmission or reflectance configuration with a number of optical source/detector pairs limited only by the size and coupling to the single detector array. The CTOT imaging system may employ a continuous wave (CW) measurement approach that is simple and low-cost. The performance of the CTOT imaging system has been verified on both a tissue-mimicking phantom and an awake infant. Thus, the CTOT imaging system enables the study of brain networks in awake children.

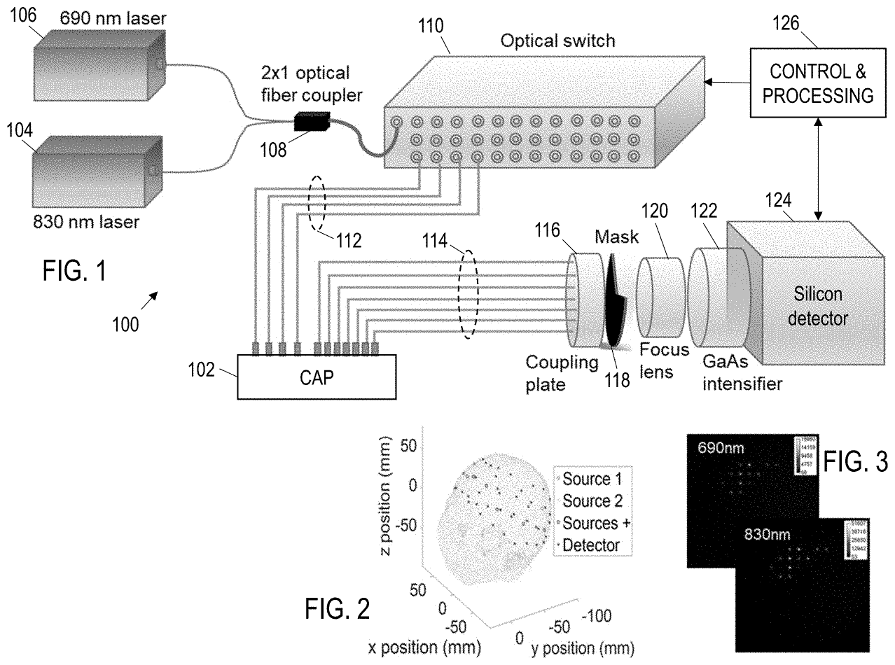

FIG. 1 shows a schematic level diagram of an example CTOT imaging system 100 in accordance with the present disclosure. The CTOT imaging system 100 includes a cap 102, a laser 104, a laser 106, an optical coupler 108, an optical switch 110, source optical fibers 112, collection optical fibers 114, a coupling plate 116, a dynamic mask 118, a lens 120, an intensifier 122, an image detector 124, and control and processing circuitry 126. The laser 104 is a laser source that generates NIR light of a first wavelength (e.g., 830 nanometer wavelength), and the laser 106 is a laser source that generates NIR light of a second wavelength (e.g., 690 nanometer wavelength). In some implementations the laser 104 and the laser 106 may respectively by an HPD 1305-9mm-83005, and an HPD 1305-9mm-69005 by Intense, North Brunswick, N.J. Other laser source and/or wavelengths may be used in some implementations of the CTOT imaging system 100.

The laser 104 is coupled by a first optical fiber to the optical coupler 108, and the laser 106 is coupled by a second optical fiber to the optical coupler 108. Activation of the laser 104 or the laser 106 may be controlled by the control and processing circuitry 126. The optical coupler 108 is coupled to the optical switch 110 by a third optical fiber to provide light generated by the laser 104 or the laser 106 to the optical switch 110. In some implementations, the laser 104 and the laser 106 may be directly coupled to the optical switch 110.

The optical switch 110 switches light received from the laser 104 or the laser 106 to the source optical fibers 112 for routing to the cap 102. While FIG. 1 shows that the source optical fibers 112 includes four optical fibers, various implementations of the source optical fibers 112 may include more than four optical fibers (e.g., 8 optical fibers, 16, optical fibers, etc.). The routing provided by the optical switch 110 may be controlled by the control and processing circuitry 126. The optical switch may be an LT1100 by LIGHTech of San Leandro, Calif. in some implementations of the CTOT imaging system 100. Other light switches may be used in some implementations of the CTOT imaging system 100.

The source optical fibers 112 and the collection optical fibers 114 are terminated in the cap 102. The cap 102 is a head covering that directs light received via the source optical fibers 112 to the head of a subject (e.g., an infant). The cap 102 may be formed of plastic or other materials suitable for terminating the source optical fibers 112 and the collection optical fibers 114 and engaging the head of a subject to hold the source optical fibers 112 and the collection optical fibers 114 in place.

The collection optical fibers 114 transmit light from the from the cap 102 to the coupling plate 116. That is, the collection optical fibers 114 transmit light illuminating (e.g., passing through) the head of a subject (i.e., light provided by the source optical fibers 112) from the cap 102 to the coupling plate 116. The collection optical fibers 114 may include any number of optical fibers (e.g., 40-50 optical fibers, 50-100 optical fibers, etc.). The coupling plate 116 conveys light incident on an input surface of the coupling plate 116 (i.e., light output of the collection optical fibers 114) to an output surface of the coupling plate 116. The coupling plate 116 may include a large number of micron diameter optical fibers that couple the input surface to the output surface.

The mask 118 may be a movable filter disposed on the output surface of the coupling plate 116. The mask 118 includes an opaque portion to block a first portion of the light received from the cap 102, and a transparent portion to pass a second portion of the light received from the cap 102. The positioning of the mask 118 may be controlled by an electric motor (not shown in FIG. 1), which is in turn controlled by the control and processing circuitry 126. Because the dynamic range of the image detector 124 limits the accurate simultaneous collection of the range of fluence output from all collection optical fibers 114, the mask 118 is positioned on the coupling plate 116 to block strong signals, to prevent saturation of the image detector 124 at high gains. Thus, for laser light provided by a given source fiber, the mask 118 may block light received from collection fibers disposed within a predetermined range of the source fiber in the cap 102. Other embodiments of the mask can include liquid crystal variable attenuators or digital micromirrors to filter fluences that exceed the dynamic range of the detector.

The lens 120 receives the image from the coupling plate 116 as filtered by the mask 118, and focuses the image on the intensifier 122 (gallium arsenide (GaAs) intensifier 122). The lens 120 may be an AF NIKKOR 28 mm f/2.8D by Nikon, NY in some implementations of the CTOT imaging system 100. A different lens may be used in some implementations of the CTOT imaging system 100.

The intensifier 122 may be a generation 3 thin film image intensifier. The intensifier 122 converts low levels of light received from the cap 102 to higher levels of light. At a photocathode of the intensifier 12, NIR photons received from the lens 120 are converted to electrons which are amplified (e.g., amplified by 10⁶) before striking a phosphor screen to generate light at a higher intensity than was received. In one implementation of the CTOT imaging system 100, the intensifier 122 may be a model: 273686, ITT Night Vision, Roanoke, Va. Some implementations of the CTOT imaging system 100 may include a different intensifier.

Light amplified by the intensifier 122 is integrated at the image detector 124. The image detector 124 may be a charge coupled device (CCD) (e.g., a 16-bit CCD) in some implementations of the CTOT imaging system 100. In some implementations, the image detector 124 may be a complementary metal oxide semiconductor (CMOS) image detector to increase image capture speed. The image detector 124 may be an e2v CCD47-20 back-illuminated chip (Princeton Instruments, Trenton, N.J.) in some implementations of the CTOT imaging system 100. Some implementations of the CTOT imaging system 100 may include a different image detector.

Images captured by the image detector 124 are transferred to the control and processing circuitry 126 for processing. The control and processing circuitry 126 controls the laser 104, the laser 106, the optical switch 110, and the mask 118 (e.g., via an electric motor) to generate images at the image detector 124, and processes the images captured by the image detector 124 to generate functional brain information. Under the control of the image detector 124, images of the collection optical fibers 114 output are acquired at each wavelength in response to illumination by each optical fiber of the source optical fibers 112. From the images, the output fluence at each optical fiber of the collection optical fibers 114 is used as input into an inversion algorithm to obtain the functional information of investigated tissues. For example, images of absorption and the concentrations of oxygenated hemoglobin (HbO), de-oxygenated hemoglobin (HbR), and total hemoglobin (HbT), as well as oxygen saturation (SO₂) may be reconstructed using NIRFAST, a modeling and image reconstruction package (or other image processing software), with a reconstruction pixel basis of 25×25×20 (x, y and z spatial discretization) uniform cells and a soft constrain. Axial, Sagittal and Coronal views of the 3-D reconstructed absorption and chromophores may be plotted using NIRFAST Slicer 1.0 software (Open source software for multi-modal optical molecular imaging) (or other image processing software).

The control and processing circuitry 126 may be implemented using a computer, such as a desktop computer, a laptop computer, a rackmount computer, an embedded computer, or other computing device that includes a processor to execute instructions read from a memory coupled to the processor. The memory is a non-transitory computer-readable medium that stores instructions performing the various functions disclosed herein. For example, the memory may store instructions that are executed by the processor to control the laser 104, the laser 106, the optical switch 110, the mask 118 and/or the image detector 124 as disclosed herein, and/or to process images captured by the image detector 124 as disclosed herein.

Various implementations of the CTOT imaging system 100 may use time-independent CW measurements due to their simplicity, or time-dependent, frequency-domain measurement of phase and amplitude.

FIG. 2 shows an example of placement of a plurality of source optical fibers and a plurality of collection optical fibers via the cap 102 on the head of a subject to be imaged using CTOT imaging system 100.

FIG. 3 shows an example of images of a coupling plate 116 showing fluence from the collection optical fibers 114 with illumination by 690 nm and 830 nm laser light in the CTOT imaging system 100;

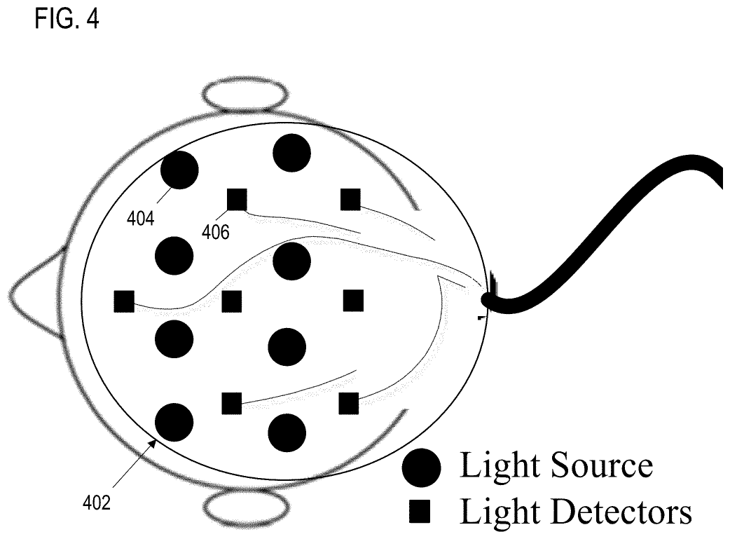

FIG. 4 shows an example of cap 402 including source fibers 406 and collection fibers 404 disposed for imaging in accordance with the present disclosure. Implementations of the 402 may include any number of source fibers 406 and collection fibers 404.

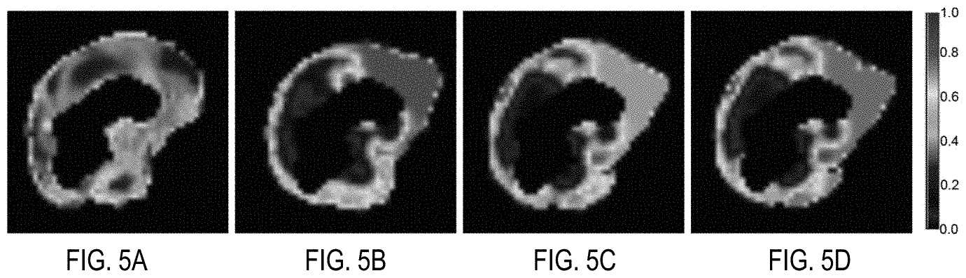

FIGS. 5A-5D shows a selection of images generated by fMRI and CTOT in accordance with the present disclosure. FIG. 5A shows a sagittal slice of the one minus the normalized intensity processed BOLD image acquired using fMRI. FIG. 5B shows a sagittal slice of a normalized intensity CTOT-HbR image. FIG. 5C shows a sagittal slice of the normalized intensity CTOT-HbR image. FIG. 5D shows a sagittal slice of the normalized intensity CTOT-HbT image. The BOLD image is strongly correlated with the CTOT images.

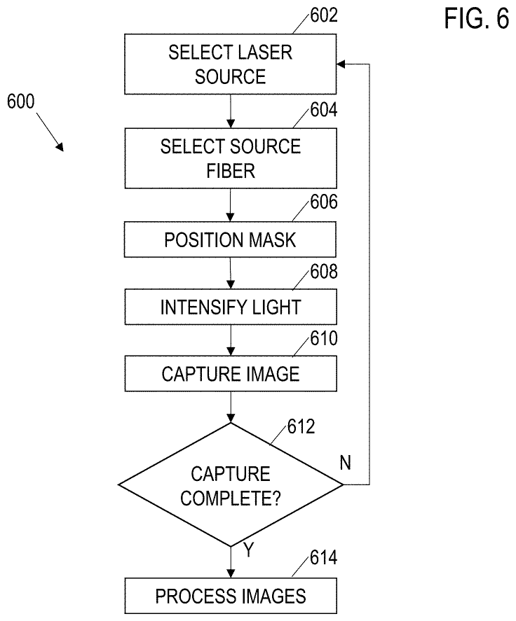

FIG. 6 shows a flow diagram for a method 600 for CTOT in accordance with the present disclosure, Though depicted sequentially as a matter of convenience, at least some of the actions shown can be performed in a different order and/or performed in parallel. Additionally, some implementations may perform only some of the actions shown. In the method 600, the cap 102 has been disposed on the head of subject to be imaged. The subject may be actively moving as the various operations of the method 600 are performed.

In block 602, the control and processing circuitry 126 selects and enables the laser 104 or the laser 106 to generate laser light. Laser light generated by the enabled laser is routed to the optical switch 110 via the optical coupler 108.

In block 604, the control and processing circuitry 126 configures the optical switch 110 to route the laser light received from the laser 104 or the laser 106 to a selected optical fiber of the source optical fibers 112.

In block 606, image detector 124 positions the mask 118 based on the optical fiber of the source optical fibers 112 selected in block 604. The image detector 124 positions the mask 118 to block light received from optical fibers of the collection optical fibers 114 that are disposed in the cap 102 near the optical fiber of the source optical fibers 112 selected in block 604.

In block 608, the intensifier 122 intensifies the light received from the from the cap 102 through the mask 118.

In block 610, the image detector 124 captures an image of the intensified light generated by the intensifier 122.

In block 612, if images have been captured at all desired laser wavelengths and with illumination via all desired source optical fibers 112, then the method 600 continues to process the captured images in block 614 and generate functional brain information.

If, in block 612, images have not been captured at all desired laser wavelengths and with illumination via all desired source optical fibers 112, then the method 600 continues with illumination via a selected laser source and source fiber in block 602.

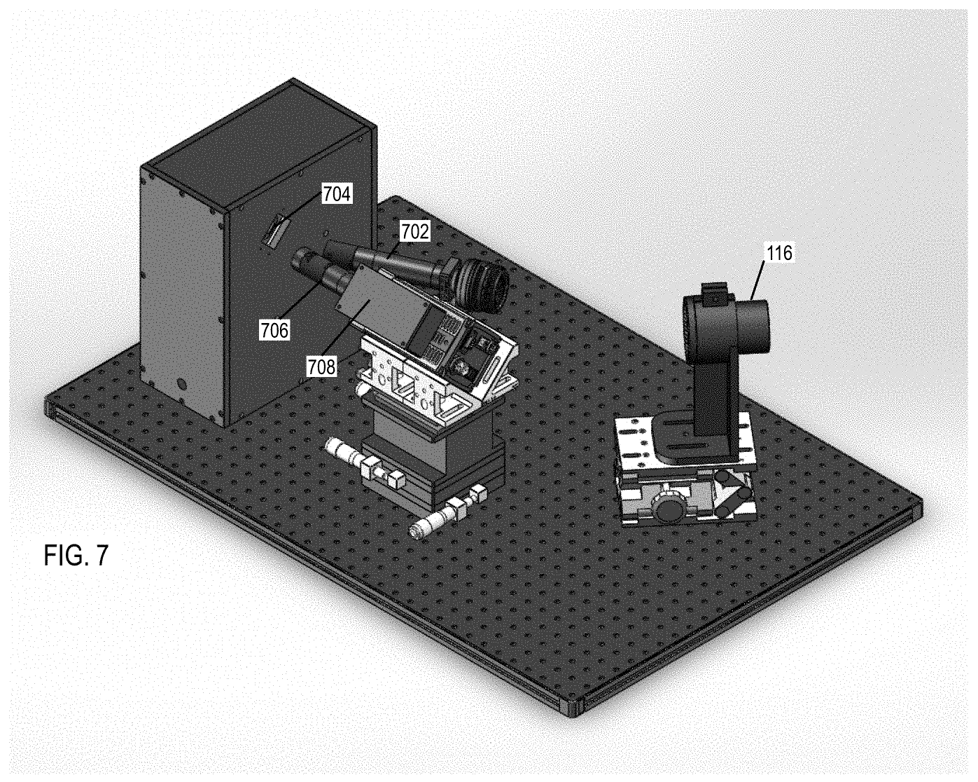

FIG. 7 shows application of a digital micromirror device (DMD) in the CTOT imaging system 100. In FIG. 7, the intensifier 122 and image detector 124 are implemented as an intensified scientific CMOS (IsCMOS) camera 708. The dynamic mask 118 of the CTOT imaging system 100 is implemented by the DMD 704. The coupling plate 116 is imaged on the surface of the DMD 704 by the lens 702. Each mirror of the DMD 704 is imaged on a corresponding pixel or bin of pixels of the IsCMOS camera 708 by the lens 706.

The DMD 704 acts as a dynamic optical switch to rapidly control the delivery of light to the intensifier photocathode of the IsCMOS camera 708. The DMD 704 includes a two-dimensional array of micromirrors (e.g., hundreds of thousands of individually actuatable microscopic mirrors) that can be individually rotated (e.g., +/−12°) to an “on” or “off” state within several hundred microseconds, effectively acting as a shutter to each pixel or bin of pixels on the detector array. To use the DMD 704 to avoid detector saturation, the control and processing circuitry 126 provides program specific “on” and “off” patterns to the DMD 704 that correspond to collecting fibers located far and near, respectively to the source fiber 112 delivering light. Therefore, the DMD 704 will direct which collection fiber light 114 enters the IsCMOS camera 708 and as such, the DMD 704 can also be utilized as a “gain control” device whereas gain of the multichannel plate (MCP) and integration time can be increased when collection of light occurs far from a source fiber 112, and decreased when collection of light occurs close to a source fiber 112. In this manner the dynamic range limitation of the IsCMOS camera 708 will be avoided enabling transcranial measurements as may be accomplished via the mask 118.

The above discussion is meant to be illustrative of the principles and various embodiments of the present invention. Numerous variations and modifications will become apparent to those skilled in the art once the above disclosure is fully appreciated. It is intended that the following claims be interpreted to embrace all such variations and modifications.

What is claimed is:

1. A cap-based Transcranial Optical Tomography (CTOT) imaging system, comprising:
a cap configured for placement on a head of a subject to be imaged;
a first laser source configured to generate a laser light at a first wavelength;
a second laser source configured to generate laser light at a second wavelength;
a first optical fiber coupling the first laser source and the second laser to the cap;
a second optical fiber coupling the first laser source and the second laser source to the cap;
an image intensifier;
a third optical fiber coupling the image intensifier to the cap;
a fourth optical fiber coupling the image intensifier to the cap; and
an image sensor coupled to the image intensifier, and configured to capture an image of intensified light generated by the image intensifier.

2. The CTOT imaging system of claim 1, further comprising control and processing circuitry coupled to the image sensor, and configured to process images captured by the image sensor.

3. The CTOT imaging system of claim 2, further comprising a coupling plate configured to convey light from the third optical fiber and the fourth optical fiber to the image intensifier.

4. The CTOT imaging system of claim 3, further comprising a lens configured to focus light from the coupling plate on the image intensifier.

5. The CTOT imaging system of claim 3, further comprising a moveable mask disposed between the coupling plate and the image intensifier, and comprising:
an opaque portion configured to block a portion of the light from the coupling plate; and
a transparent portion configured to pass a second portion of the light from the coupling plate.

6. The CTOT imaging system of claim 5, further comprising a motor coupled to the moveable mask and configured to control the position of the moveable mask; wherein the control and processing circuitry is coupled to the motor and configured to control the motor for positioning the moveable mask.

7. The CTOT imaging system of claim 3, further comprising a digital micromirror device configured to reflect light from the coupling plate to the image intensifier; wherein the control and processing circuitry is coupled to the digital micromirror device and configured to control the positioning of micromirrors of the digital micromirror device.

8. The CTOT imaging system of claim 7, further comprising a lens configured to focus light from the coupling plate on the digital micromirror device.

9. The CTOT imaging system of claim 2, further comprising an optical switch configured to:
route the laser light generated by the first laser source to the first optical fiber and the second optical fiber; and
route the laser light generated by the second laser source to the first optical fiber and the second optical fiber;
wherein the control and processing circuitry is coupled to the optical switch and configured routing of the laser light generated by the first laser source and routing of laser light generated by the second laser source.

10. A method for cap-based Transcranial Optical Tomography (CTOT) comprises:
placing a cap on a subject to be imaged;
selecting a laser source of a plurality of available laser sources to generate laser light;
selecting a source fiber or a plurality of available source fibers to route the laser light to the cap;
filtering light received from the cap through a first collection fiber positioned within a predetermined distance of the source fiber routing laser light to the cap; and
capturing an image of light received from the cap through a second collection fiber positioned more than the predetermined distance from the source fiber routing laser light to the cap.

11. The method of claim 10, further comprising conveying the light received through the first collection fiber and the light received through the second collection fiber through a coupling plate.

12. The method of claim 11, wherein filtering the light comprises activating a motor to position a moveable mask disposed adjacent an output surface of the coupling plate; wherein the motor positions the moveable mask to block the light received through the first collection fiber and pass the light received through the second collection fiber.

13. The method of claim 11, wherein filtering the light comprises setting micromirrors of a digital micromirror device to block the light received through the first collection fiber and pass the light received through the second collection fiber.

14. The method of claim 13, further comprising focusing the light conveyed by the coupling plate on the digital micromirror device.

15. The method of claim 10, further comprising focusing the light received from the cap through a second collection fiber on an intensifier.

16. The method of claim 15, wherein capturing the image comprises capturing an image of intensified light produced by the intensifier.

17. The method of claim 10, further comprising processing the image to generate functional brain information.

18. A cap-based Transcranial Optical Tomography (CTOT) imaging system, comprising:
a first laser source configured to generate a first wavelength of laser light;
a second laser source configured to generate a second wavelength of laser light;
an optical switch configured to receive the laser light generated by the first laser source and the laser light generated by the second laser source;
a plurality of source optical fibers coupled to the optical switch;
a cap coupled to the plurality of source optical fibers;
a plurality of collection optical fibers coupled to the cap;
a coupling plate coupled to the plurality of collection optical fibers;
a mask configured to receive light passed through the coupling plate;
an image sensor configured to capture an image of light passed by the mask; and
control and processing circuitry configured to:
control activation of the first laser source and the second laser source;
control routing, by the optical switch, of the laser light generated by the first laser source and the laser light generated by the second laser source to the plurality of source optical fibers; and
control blocking and passing of light by the mask.

19. The CTOT imaging system of claim 18, wherein the control and processing circuitry is configured to set the mask to block light received from a collection optical fiber positioned, in the cap within a predetermined distance of a source fiber providing laser light to the cap.

20. The CTOT imaging system of claim 18, further comprising:
an intensifier; and
a lens configured to focus light passed by the mask on the intensifier;
wherein the image sensor is configured to capture an image of intensified light generated by the intensifier.
